(19) United States
(12) Patent Application Publication
(10) Pub. No.: US 2016/0178712 A1
(43) Pub. Date: Jun. 23, 2016

(54) SWITCHING FOR DISTRIBUTING RECEIVE SIGNALS OF AN MR SYSTEM TO RECEIVERS

(71) Applicant: Stephan Biber, Erlangen (DE)

(72) Inventor: Stephan Biber, Erlangen (DE)

(21) Appl. No.: 14/978,775

(22) Filed: Dec. 22, 2015

(30) Foreign Application Priority Data
Dec. 23, 2014 (DE) 102014226947.4

(51) Int. Cl. G01R 33/34; G01R 33/30; G01R 33/385; A61B 5/055

(52) U.S. Cl. CPC G01R 33/34046; A61B 5/0555; G01R 33/30; G01R 33/385; G01R 33/34069

(57) ABSTRACT

The present embodiments relate to a magnetic resonance tomography system including a plurality of MRT connectors for connecting antenna elements of one of a plurality of local coils. The antenna elements of one of the local coils connected to one of the MRT connectors may be switched by a switching matrix to receivers of one of a plurality of receiver blocks, each having a plurality of receivers.

### CROSS-REFERENCE TO RELATED APPLICATIONS

The present patent document claims the benefit of DE 102014226947.4, filed on Dec. 23, 2014, which is hereby incorporated by reference in its entirety.

### TECHNICAL FIELD

The present embodiments relate to magnetic resonance tomography devices.

### BACKGROUND

Magnetic resonance devices (MRTs) for examining objects or patients by magnetic resonance tomography are known (e.g., DE 103 14 215 B4, DE 10 2013 019 643.4 and CN 2013 20574475.2).

### SUMMARY

The scope of the present invention is defined solely by the appended claims and is not affected to any degree by the statements within this summary

The present embodiments may obviate one or more of the drawbacks or limitations in the related art. For example, the present embodiments optimize a magnetic resonance tomography system.

### BRIEF DESCRIPTION OF THE DRAWINGS

FIG. 1 depicts an embodiment of a patient couch having patient couch connectors at the head end and at the foot end.

FIG. 2 depicts example patient couch connectors at the head end of a patient couch, a switching matrix and receivers.

FIG. 3 depicts an embodiment of patient couch connectors at the head end of a patient couch, a switching matrix and two blocks of receivers on the left, and six different use cases/settings/switch positions of the switching matrix on the right.

FIG. 4 depicts an embodiment of patient couch connectors at the head end of a patient couch, a switching matrix and two blocks of receivers on the left, and four different use cases/settings/switch positions of the switching matrix on the right.

FIG. 5 depicts an embodiment of patient couch connectors at the head end of a patient couch, a switching matrix and three blocks of receivers.

FIG. 6 depicts an embodiment of patient couch connectors at the head end of a patient couch, a switching matrix and three blocks of receivers in seven different use cases/settings/switch positions of the switching matrix.

FIG. 7 depicts an embodiment of transmission of signals received by antenna elements of a local coil via connectors and receivers in a patient table to an evaluation device of an MRT.

FIG. 8 depicts a schematic of an MRT system.

### DETAILED DESCRIPTION

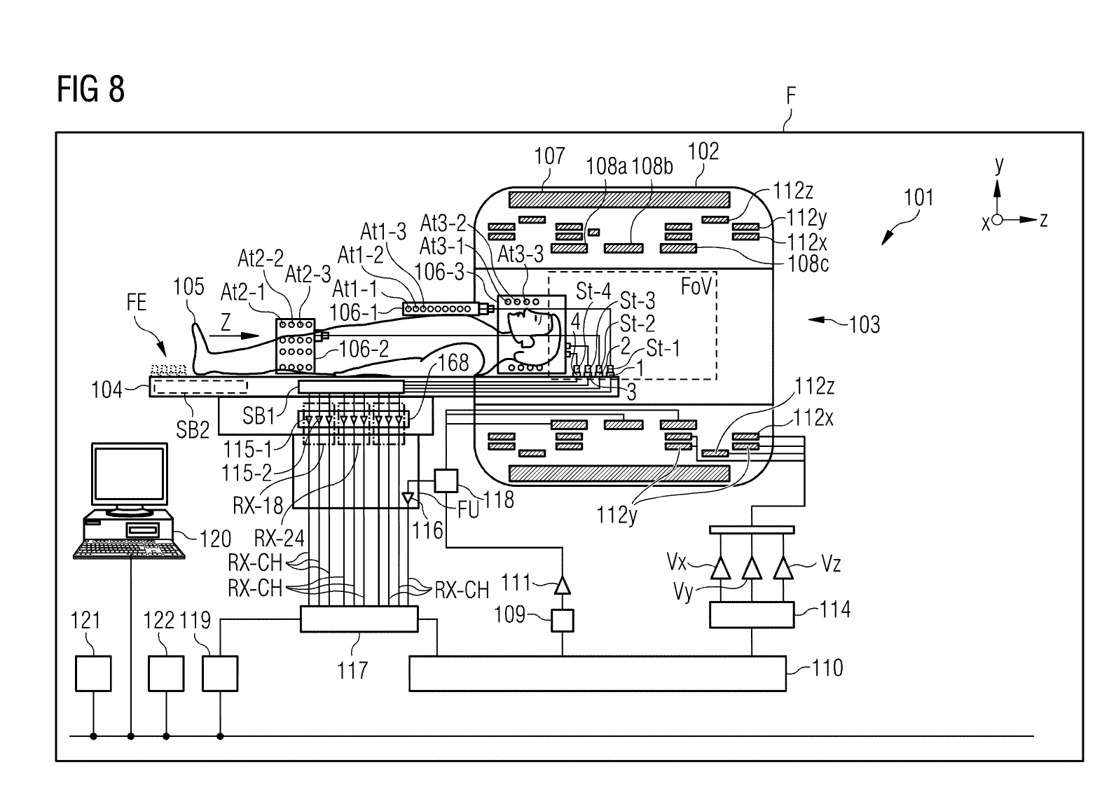

FIG. 8 depicts, among other things, an imaging magnetic resonance device MRT 101 (e.g., contained in a shielded room or Faraday cage F) having a housing 102 in the shape of a hollow cylinder with an in this case tubular chamber 103 into which a patient couch 104 supporting a body (e.g., of an examination subject, such as a patient) 105 (e.g., with or without local coil arrangement 106-1, 106-2, and 106-3) may be moved in the direction of the arrow z in order to generate images of the patient 105 by an imaging method. Disposed on the patient 105 is a local coil arrangement 106 with which it is possible, in a local region (e.g., field of view or FoV) of the MRT, to generate images of a subregion of the body 105 in the FoV. Signals of the local coil arrangement 106 may be evaluated (e.g., converted into images, stored or displayed) by an evaluation device (e.g., 168, 115, 117, 119, 120, 121, etc.) of the MRT 101 that may be connected to the local coil arrangement 106 (e.g., by way of coaxial cable or wirelessly (167), etc.).

In order to examine a body 105 (e.g., an examination subject or a patient) by a magnetic resonance imaging scan using a magnetic resonance device MRT 101, different magnetic fields that are correlated with one another with the utmost precision in terms of their temporal and spatial characteristics are radiated onto the body 105. A powerful magnet (e.g., often a cryomagnet 107) in a measurement cabin having a tunnel-shaped opening or bore 103 generates a strong static main magnetic field B₀ (e.g., ranging from 0.2 tesla up to 3 tesla or more). A body 105 that is to be examined, supported on a patient couch 104, is moved into position in a region of the main magnetic field Bo that is approximately homogeneous in the area under observation FoV. The nuclear spins of atomic nuclei of the body 105 are excited by magnetic radiofrequency excitation pulses B1(x, y, z, t) that are radiated in by way of a radiofrequency antenna and represented in a highly simplified form as a (e.g., 108a, 108b, and 108c) body coil 108 (e.g., where appropriate, by a local coil arrangement). Radiofrequency excitation pulses are generated (e.g., by a pulse generation unit 109 that is controlled by a pulse sequence control unit 110). Following amplification by a radiofrequency amplifier 111, the RF excitation pulses are routed to the radiofrequency antenna 108. The radiofrequency system is depicted only schematically. It is also possible for more than one pulse generation unit 109, more than one radiofrequency amplifier 111, and a plurality of radiofrequency antennas 108a, 108b, and 108c to be used in a magnetic resonance device 101.

The magnetic resonance device 101 includes gradient coils 112x, 112y, and 112z by which magnetic gradient fields B_{G}(x, y, z, t) are radiated during a measurement for the purpose of selective slice excitation and for spatially encoding the measured signal. The gradient coils 112x, 112y, and 112z are controlled by a gradient coil control unit 114 (e.g., and if necessary by amplifiers Vx, Vy, and Vz), that, like the pulse generation unit 109, is connected to the pulse sequence control unit 110.

Signals emitted by the excited nuclear spins (e.g., the atomic nuclei in the examination subject) are received by the body coil 108 and/or at least one local coil arrangement 106, amplified by associated radiofrequency preamplifiers 116, and processed further and digitized by a receive unit 117. The recorded measurement data is digitized and stored as complex numeric values in a k-space matrix. An associated MR image may be reconstructed from the value-filled k-space matrix by a multidimensional Fourier transform.

For a coil that may be operated in both transmit and receive mode (e.g., the body coil 108 or a local coil 106-1, 106-2, and 106-3), correct signal forwarding is controlled by an upstream-connected duplexer 118.

Based on the measurement data, an image processing unit 119 generates an image that is presented for viewing by a user by an operator console 120 and/or stored in a memory unit 121. A central processing unit 122 controls the individual system components.

For example, local coil arrangement 106 (e.g., 106-1, 106-2, and 106-3) is the term generally applied to an antenna system that may include one antenna element or an array coil (e.g., a plurality of antenna elements At1-1-At1-3, At2-1-At2-3, and At3-1-At3-3). Said individual antenna elements At1-1-At1-3, At2-1-At2-3, and At3-1-At3-3 may be provided as loop antennas (e.g., loops), butterfly coils, flex coils or saddle coils. For example, a local coil arrangement may include antenna elements, a preamplifier, further electronics (e.g., standing wave traps, etc.), a housing, supports, and, in most cases, a cable with local coil connectors St-1-St-4 (e.g., by which the local coil arrangement is connected to patient couch connectors 1, 2, 5, and 6 of a patient couch 104 and/or MRT system 101). Mounted on the system side is at least one receiver (e.g., 168, RX-CH, RX24, RX18, and RX6). Each of the receivers has one amplifier or, as in this case, a plurality of amplifiers (e.g., 115-1, 115-2, etc. are represented by a group RX18 of 18 receivers RX-CH and a group RX24 having 24 receivers RX-CH; or RX24, RX18, RX6 and/or amplifiers). The receivers filter and digitize a signal received by a local coil 106-1, 106-2, and 106-3 (e.g., via cable), etc., and transfer the data to a digital signal processing device, which in most cases, derive an image or a spectrum from the data obtained by a measurement and make the image or spectrum available to the user (e.g., for subsequent diagnosis by the user and/or for storage).

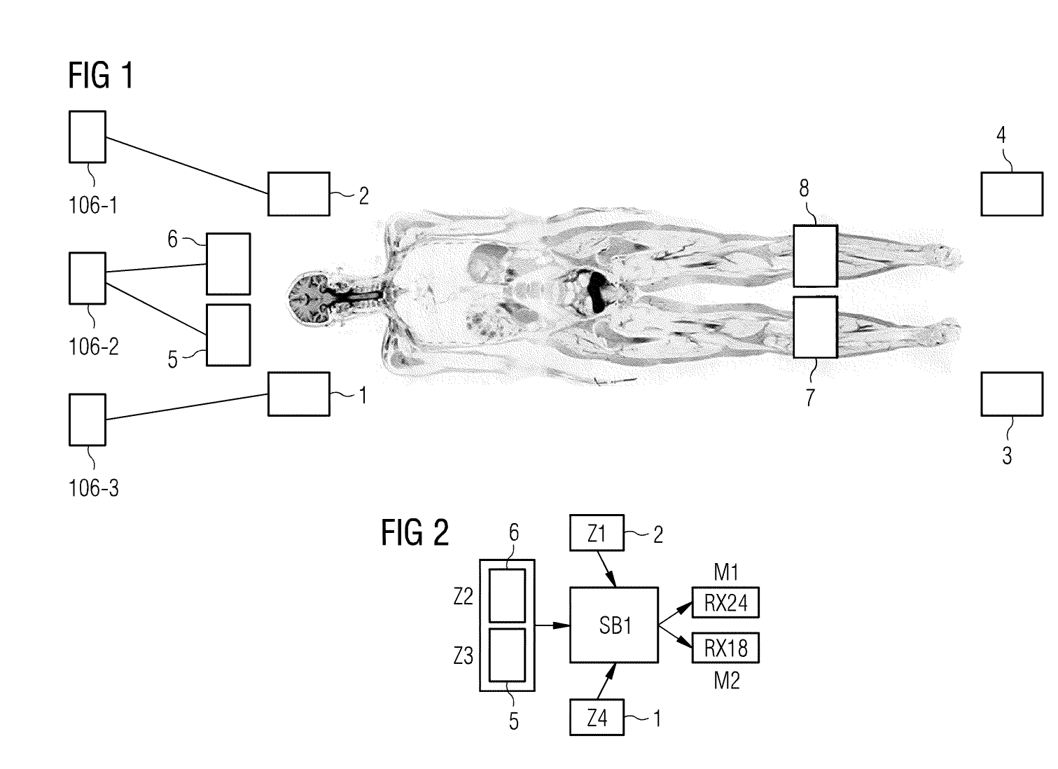

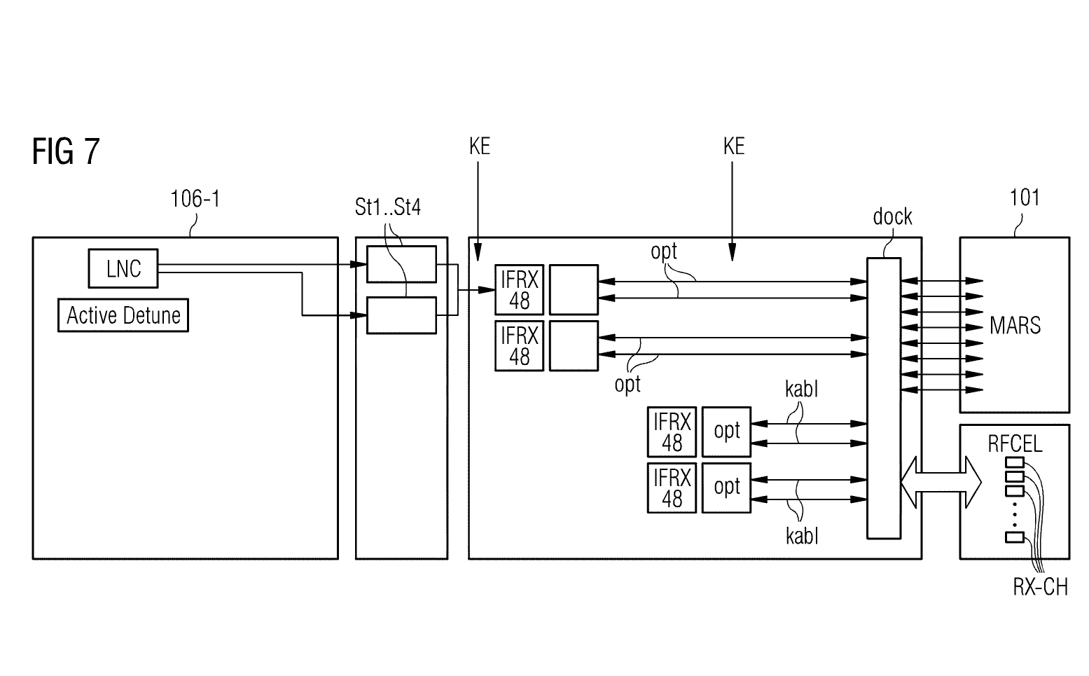

FIGS. 1, 7 and 8 depict, in simplified form, images having a high signal-to-noise ratio that are generally recorded in present-day MR tomography of components called local coils (e.g., loops) 106-1, 106-2, and 106-3.

The excited nuclei induce a voltage in antenna elements At1-1-At1-3, At2-1-At2-3, and At3-1-At3-3 of a local coil 106-1, 106-2, and/or 106-3, said voltage then being amplified (e.g., by a low-noise preamplifier LNA in a respective local coil). For example, the voltage is forwarded (e.g., by cable at the MR frequency) via local coil connectors St1-St4 and head-end (KE) S MRT connectors (e.g., patient couch connectors on the patient couch) 1, 2, 5, and 6 and optical connections opt, hardwired connections (kab), and/or a docking station (DOCK) in a patient couch 104 to receivers RX-CH (e.g., having receive electronics RFCEL and/or amplifiers, etc.) in T receive blocks RX24, RX18, and RX6 of a magnetic resonance tomography system 101.

Systems (e.g., known as high-field systems) are used to improve the signal-to-ratio (e.g., including high-resolution images). Currently, such systems have basic field strengths around 3 tesla and higher. Considering the intention to enable more antenna elements At1-1-At1-3, At2-1-At2-3, and At3-1-At3-3 (e.g., “loops,” “antenna element,” “antenna,” or “coil element”) to be connected to a MR receive system than there are receivers RX-CH (e.g., receive electronics channels) available, a switching matrix (e.g., also called an RCCS or “Receive-Coil Channel Selector”) is installed between the receive antennas and receivers RX-CH. The switching matrix routes the currently active local coil antennas (e.g., of the active local coils 106-1, 106-2, and 106-3) to the available receivers RX-CH of the MRT.

Thus, it may be possible to connect more antenna elements At1-1-At1-3, At2-1-At2-3, and At3-1-At3-3 than there are receivers RX-CH (e.g., in receiver blocks RX24, RX18, and/or RX6), because with a whole-body coverage it may be necessary to read out only those antenna elements (e.g., of local coils 106-1, 106-2, and 106-3) that are located in the FoV (e.g., Field of View) or, as in this embodiment, in the homogeneity volume of the magnet.

In the following description, the term “local coil” or “coil” may refer to an antenna that may consist of one or more antenna elements At1-1-At1-3, At2-1-At2-3, and At3-1-At3-3 (e.g., loops and or as an array coil). For example, a local coil 106-1, 106-2, and/or 106-4 may contain antenna elements At1-1-At1-3, At2-1-At2-3, and At3-1-At3-3, a preamplifier, further electronics and cabling, a housing, and, in most cases, at least one cable having a local coil (e.g., cable) connector St-1 or St-2, or multiple local coil (e.g., cable) connectors St-3 and St-4 (e.g., in the case of multiple cables), by which it is connected to the MRT system (e.g., an “MRT system” is also understood in this context an MR(T) device 101).

In an MR receive system, the antenna elements At1-1-At1-3, At2-1-At2-3, and At3-1-At3-3 may be connected (e.g., in a distributed manner) to the individual receivers RX-CH, RX24, RX18, and/or RX6. For example, because more local coils 106-1, 106-2, and/or 106-3, and/or antenna elements At1-1-At1-3, At2-1-At2-3, and At3-1-At3-3 may be placed on a patient 105 than are located simultaneously in the field of view (FoV) of the MRT magnet, it may be beneficial to operate fewer receivers RX-CH than antenna elements At1-1-At1-3, At2-1-At2-3, and At3-1-At3-3. For example, also suitable for this purpose is at least one switching matrix SB1 (e.g., and/or SB2) that may route the antenna elements At1-1-At1-3, At2-1-At2-3, and At3-1-At3-3 that can be connected to the patient table (e.g., “PTAB” 104) in a flexible manner to the available receivers RX-CH of the magnetic resonance tomography system 101.

Antenna elements At1-1-At1-3, At2-1-At2-3, and At3-1-At3-3 of one or more local coils 106-1, 106-2, and/or 106-3 that are connected via one (St-1 or St-2) or more (St-3 and St-4) local coil (e.g., cable) connectors on MRT connectors (e.g., MRT connectors) 1, 2, 5, and 6 (e.g., “coil sockets”) are routed to the available receivers RX-CH (e.g., in blocks RX24, RX18, and RX6 in the MRT 101).

Signals (e.g., received by at least one antenna element At1-1-At1-3, At2-1-At2-3, and At3-1-At3-3 of a local coil 106-1, 106-2, and/or 106-3) are routed (e.g., on physically separate cables and/or frequencies inside a cable and/or a local coil (e.g., cable) connector St-1, St-2, St-3 and St-4). A cable may transport one or more received signals RX (e.g., signals of one or more antenna elements At1-1-At1-3, At2-1-At2-3, and At3-1-At3-3).

When the individual physical cables inside a local coil connector (e.g., St-1, St-2, St-3 and St-4) are referenced, reference is also made to “coax connectors” in order to differentiate the terms without necessarily having to implemented as coaxial cables or connectors. However connectors may be implemented as coaxial cables or connectors according to one embodiment.

For example, one embodiment of a receive system of an MRT differentiates between two capacity-indicating numbers M and N. M refers to the number of channels of antenna elements At1-1-At1-3, At2-1-At2-3, and At3-1-At3-3 of the local coil(s) 106-1, 106-2, and 106-3 that may be connected to a patient couch (e.g., in the case of three local coils 106-1, 106-2, and 106-3 each having three antenna elements At1-1-At1-3, At2-1-At2-3, and At3-1-At3-3, then the local coils have nine channels (3+3+3)). N refers to the number of simultaneously receivable signals, where M>N. For example, receivable signals are signals that may be evaluated in the receive electronics of the MRT or in receivers RX-CH (e.g., in the case of 24 channels in RX24 and 18 channels in RX18, then N will be 42 receivable signals (24+18) able to be evaluated in receivers, and in the instance of one antenna element in each case).

Between antenna elements At1-1-At1-3, At2-1-At2-3, and At3-1-At3-3 and receivers RX-CH lies a switching mechanism in the form of a switching matrix SB1 at the head end (e.g., KE) of the patient couch 104. The switching matrix SB1 selects, from M signals of the M channels of the antenna elements At1-1-At1-3, At2-1-At2-3, and At3-1-At3-3 of the local coils 106-1, 106-2, and 106-3, a subset having a maximum size N (e.g., number of receivers RX-CH).

For example, a 32-channel local coil 106-1 has two local coil connectors St-3 and St-4. In the first local coil connector St-3, twelve coaxes (e.g., coaxial cables) are assigned, each coax conducting two signals (e.g., 24 antenna elements). In the second local coil connector St-4, four coaxes, each assigned two signals, are used (e.g., of eight antenna elements). In total, the signals of thirty-two antenna elements are conducted via two local coil connectors St3 and St-4 and sixteen coaxes (e.g., coaxial cables) are therein.

In an embodiment, a large switching matrix permits any arbitrary input (e.g., of a local coil 106-1) to be routed to any arbitrary output of the switching matrix and receivers connectable thereto. This embodiment permits many possible combinations of local coils 106-1, 106-2, and 106-3, locations of the connection of the local coil(s) on the patient table 104, assignment of the coax cables inside a local coil connector St-1, St-2, St-3, St-4, and St-1-St-4, and/or patient table connector 1, 2, 5, and 6.

In another embodiment, a switching matrix as described above may only route any arbitrary input to an output, but not to an arbitrary output.

In another embodiment, a switching matrix implements only the switching combinations necessary to connect a permanently defined local coil portfolio. In this embodiment, advantages may be cost-effectiveness (e.g., by using a small number of switches), in the space requirements, and in integration in the patient couch. However, it may be for actual customer implementations (e.g., local coil portfolio, local coil combinations) to be precisely known and fully taken into account, as otherwise the use of the MR system by the switching mechanism using a switching matrix SB1 may be limited. The switching mechanisms are operated according to known embodiments near to the magnet of the magnetic resonance tomography system or outside of the shielded cabin (F) of the magnetic resonance tomography system 101.

According to an embodiment, a system is implemented in the patient table, and may result in the costs for cabling to be saved. The embodiments disclosed above may be implemented in the patient table, on the magnet, outside the shielded cabin or at some other location.

According to an embodiment, a system may be implemented only for MRT systems having relatively few connectable antenna elements At1-1-At1-3, At2-1-At2-3, and At3-1-At3-3, and the embodiments disclosed above may be implemented for MRT systems having very high numbers of connectable coil elements.

In some embodiments, multichannel MRT systems having many local coil connectors St-1-St-4 on a patient couch 104 and having many antenna elements At1-1-At1-3, At2-1-At2-3, and At3-1-At3-3 per local coil connector St-1-St-4 may be provided with an advantageous switching mechanism.

One embodiment includes integration of the receivers RX-CH or receiver blocks RX24, RX18, and RX6 (e.g., the digitization thereof) in the patient table 104 (e.g., in the patient table tabletop PT-TT moving into the bore 103 of an MRT 101). In this embodiment, for example, it is possible to transmit the receive signals (e.g., RX signals) on optical cables to the MRT system 101, saving on costs for standing wave traps in the patient couch 104 and coaxial cables.

However, in order not to equip all pluggable channels at the head end (KE) of a patient couch 104 (e.g., with certain MRTs, four MRT connectors with 24 channels results in 96 channels (4×24)) with receivers RX-CH, RX24, RX18, and RX6, a simple switching logic is connected in the form of a switching matrix SB1 between MRT connectors 1, 2, 5, and 6 and receivers RX-CH, RX24, RX18, and RX6 in the patient couch 104.

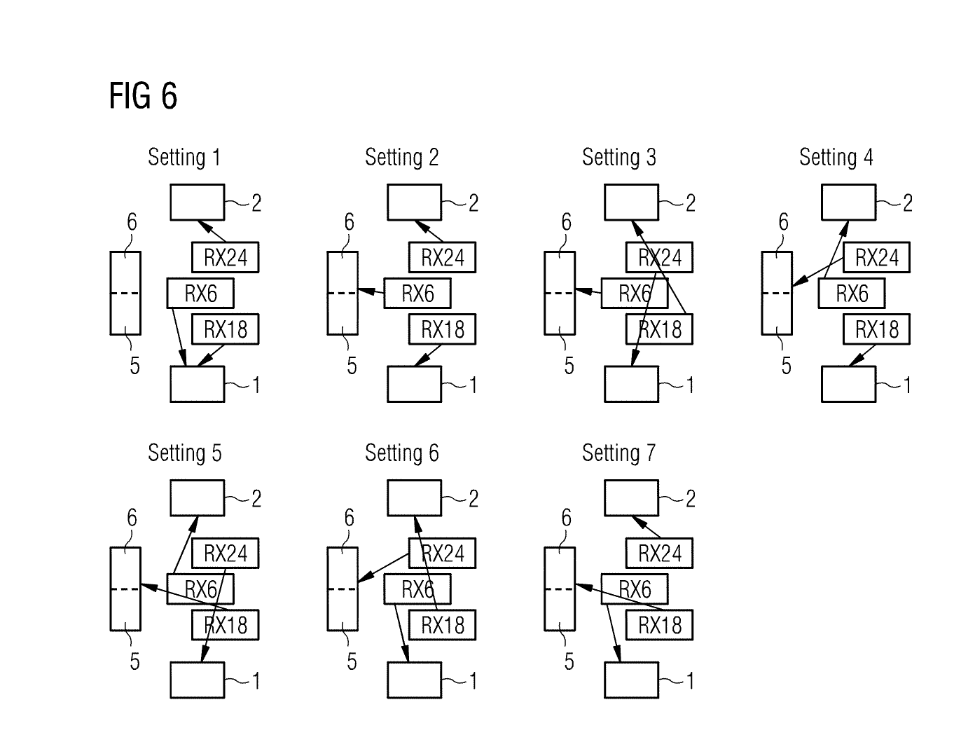

For example, FIG. 6 depicts an embodiment exemplifying an implementation that includes a 48-channel system.

According to various embodiments, located at the head end (KE) and/or the foot end (FE) of a patient couch 104 are S (e.g., four) physically separate patient couch connectors 1, 2, 5, and 6 (e.g., plug-in connections or connector sockets on a patient couch 104 for local coil connectors St1-St4 of the connecting cables of one local coil 106-1, 106-2, and/or 106-3). According to one embodiment, the patient couch connectors may include three separate patient couch connectors if, on account of the mechanical configuration, the two common (e.g., “direct connect”) connectors 5 and 6 (e.g., “direct connect” connectors also electrically connectable by plugging in) of the head local coil 106-3 are always simultaneously assigned or not assigned.

Based on a detailed use-case analysis (e.g., analysis of possible uses) with regard to the question of “how the local coils 106-1, 106-2, and 106-3 are placed on a patient couch and connected, taking into account all examinations, and which of the local coils 106-1, 106-2, and 106-3 are operated (e.g., received) simultaneously” it is apparent that normally only two or three of said four MRT connectors 1, 2, 5, and 6 are required at the head end (KE). Although local coils 106-1, 106-2, and 106-3 may be connected to all (e.g., three or four) MRT connectors 1, 2, 5, and 6. The local coils 106-1, 106-2, are 106-3 are then not located simultaneously in the field of view (FOV), with the result that only a subset of the connectable local coils 106-1, 106-2, and 106-3 must be received (e.g., digitized and forwarded) simultaneously. Detuning signals (e.g., PIN diode signals) of the antenna elements At1-1-At1-3, At2-1-At2-3, and At3-1-At3-3 are, however, used here at all times for all the connected antenna elements At1-1-At1-3, At2-1-At2-3, and At3-1-At3-3.

A logic is provided that realizes the following. A maximum of Z antenna elements At1-1-At1-3, At2-1-At2-3, and At3-1-At3-3 may be connected at the head end (KE) of a patient couch 104. The number of antenna elements At1-1-At1-3, At2-1-At2-3, and At3-1-At3-3 at one of the MRT connectors 1, 2, 5, and 6 is Zn.

With four MRT connectors, the number Z is accordingly yielded from Z=Z1+Z2+Z3+Z4; and in the case where all MRT connectors 1, 2, 5, and 6 may serve the same number (e.g., Z1=Z2=Z3=Z4) of antenna elements At1-1-At1-3, At2-1-At2-3, and At3-1-At3-3, the result obtained is Z=4*Z1 (e.g., Z1=Z2=Z3=Z4).

According to one embodiment, a maximum of M antenna elements At1-1-At1-3, At2-1-At2-3, and At3-1-At3-3 on two out of three or two out of four MRT connectors 1, 2, 5, and 6 are received simultaneously. A switching matrix having Z inputs and M outputs is connected between MRT connectors 1, 2, 5, and 6 and receivers RX-CH, RX24, RX18, and RX6. The M outputs are grouped into two subgroups M1 and M2, where M=M1+M2.

Generally, two subgroups M1 and M2 are then distributed by one switching matrix SB1 to two of the three MRT connector groups comprising MRT connectors 1, 2, 5, and 6 (e.g., to two to three of the four MRT connectors 1, 2, 5, and 6). For example, six possible switching states of the switching matrix are produced, depicted in FIGS. 2, 3, 4, 7, and 8. Where S=number of connected local coils, such as three and T=number of receiver blocks (i.e., RX24 and RX18), such as two, the number of settings is =S!/(S-T)!=3!/1!=6. Accordingly, if the receiver blocks have the same number of receivers RX-CH, the number of possible matrix settings=S!/((S-T)!*T!)=3!/(1!2!)=3.

For example, the two subgroups M1 and M2 may be chosen such that in one implementation M1=Z1 and in another implementation M2<Z1.

For example, one receiver block RX24, RX18, or RX6 (e.g., having in each case a plurality of receivers RX-CH) may serve all Z1 antenna elements of an MRT connector 1, 2, 5, and 6, and a further receiver block RX24, RX18, or RX6 only a part thereof.

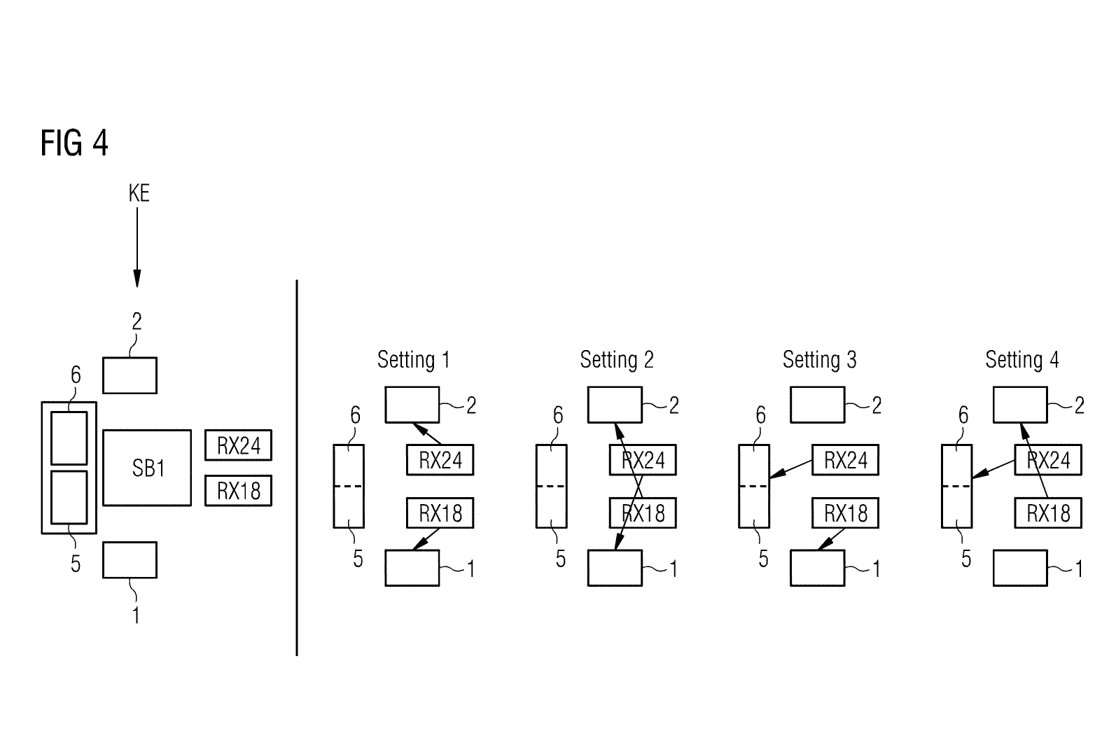

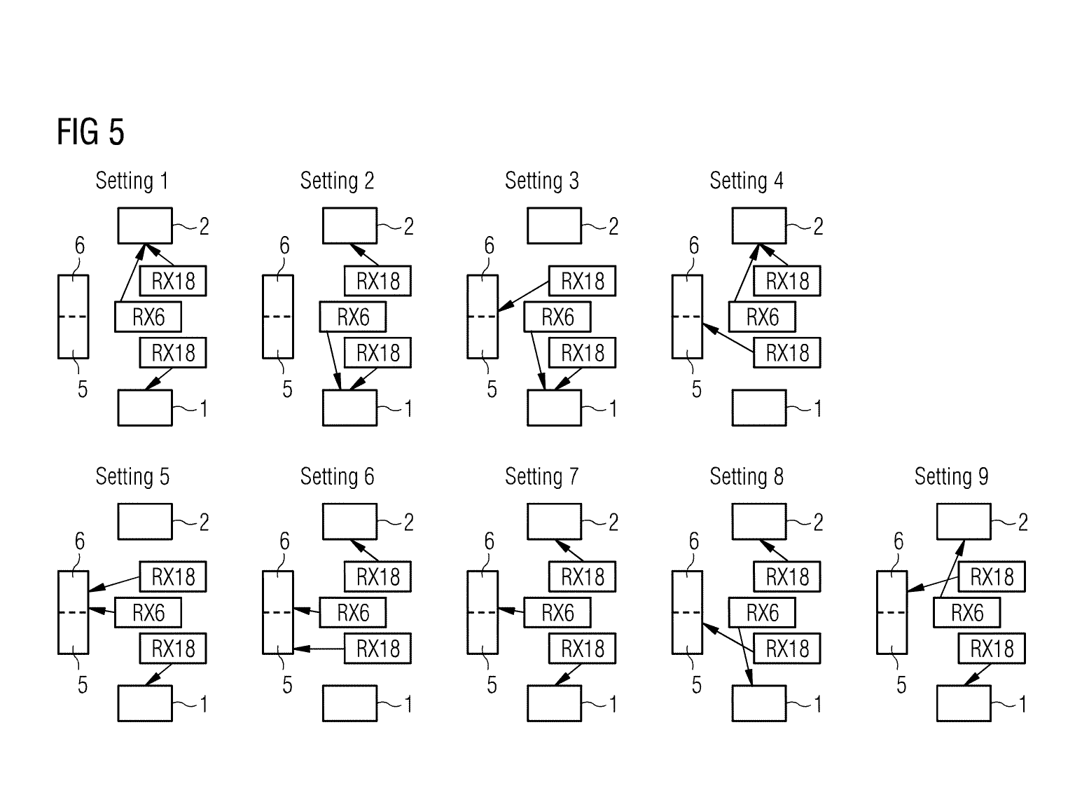

With further restrictions (e.g., from the findings of a use-case analysis), a switching matrix having only four switching states is also conceivable, as depicted in FIG. 4. For example, this embodiment may be provided by only 24 SP3T (e.g., single-pole three-throw) switches and 18 SP2T switches. FIG. 5 depicts a configuration with nine settings that distribute the available RX channels RX-CH over all three MRT connectors 1, 2, and 5. To that end, three receiver blocks RX24, RX18, and RX6 are formed, and accordingly provided that M1=18, M2=18, M3=6. The aforementioned numbers shown are exemplary embodiments only.

For example, various embodiments having three receiver blocks (e.g., RX blocks) are provided. For example, FIG. 5 depicts two blocks equal in size M1=M2 and one smaller M3<M1. FIG. 6 depicts all three blocks different in size M1<>M2<>M3. Finally, all three blocks may be equal in size M1=M2=M3.

With multiple local coils simultaneously located in a large FoV and all three received simultaneously, these configurations may enable the channels to be distributed in a favorable manner. This configuration may utilize the fact that not all three local coils are located completely in the FoV of the scanner.

Several embodiments are provided where Z1=Z2=Z3=Z4. In other words, it is assumed that all the MRT connectors may serve an equal number of antenna elements At1-1-At1-3, At2-1-At2-3, and At3-1-At3-3 (e.g., on account of the contacts contained therein).

The embodiments depicted in the figures may also be provided to MRT systems in which Z1<>Z2, etc. What is meant by Z1 in that case is the number of antenna elements At1-1-At1-3, At2-1-At2-3, and At3-1-At3-3 on that MRT connector to which the greatest number of antenna elements At1-1-At1-3, At2-1-At2-3, and At3-1-At3-3 may be connected (e.g., Z1>Zn(n<>1)).

The numbers (e.g., Z=96, M=40, M1=24, M2=18) depicted in the figures are merely exemplary embodiments.

In various embodiments, a switching matrix SB1 (e.g., and/or correspondingly SB2) selects, out of S connectors at the head end (KE), a subset T (e.g., T<S) and connects the signals contained therein wholly or partially to T receiver blocks RX24, RX18, and/or RX6 of different (channel) numbers (e.g., 24, 18, or 6) of receivers RX-CH. The number of switching matrix settings generally necessary is depicted in the figures for the embodiment of receiver blocks RX24, RX18, and RX6 (e.g., RX blocks) of different and equal size.

Further embodiments are provided for when the switching matrix SB1 is situated at the head end (KE) of the PTAB-TT, at the foot end (FE) of the patient table 104 (e.g., the PTAB-TT), or a combination thereof, and (e.g., only or also) on the housing of the magnet, at the bore 103, at the MRT 101, or in the foot (FU) of the patient table 104 (e.g., in other words, below the PTAB-TT which is the uppermost part of the patient table 104).

The Switching functions depicted may be used for patient table connectors 1, 2, 5, and 6 at the head end (KE) and/or at the foot end (FE) of a patient couch 104.

If the switching logic SB1 is not, or not only realized at the head end (KE) of the PTAB-TT of the patient table 104, it may not be possible to reduce the cable costs in the PTAB-TT of the patient table 104 to a similar degree, but it may nevertheless be possible to reduce the high costs of a current (e.g., at least internally) known RCCS switching logic implementation comprising several switches, and additionally reduce cable costs in the PTAB foot of the patient table 104.

One embodiment may also be used in combination with FDM and/or TOM transmission methods (e.g., and/or frequency-division multiplexing methods, time-division multiplexing methods, and/or code-division multiplexing methods) in the transmission from antenna elements of a local coil 106-1, 106-2, and 106-3 to at least one receiver RX-CH.

For example, various embodiments not only provide the switching functions depicted in the figures (e.g., in various combinations), but also possible subsets therefrom (e.g., in other words, only four of the nine settings are depicted in FIG. 5).

Possible advantages of certain embodiments may include one or more of the following: reduction in the number of switches required; less digital logic for controlling the switches; reduced space requirement (integration in PTAB); lower costs; lower complexity; and reduced costs of other componentry (e.g., cables).

For example, one aspect of an embodiment may lie in the realization of a switching logic (e.g., SB1 and/or SB2) tailored to the actual application for the purpose of assigning MRT connectors 1, 5, 6, and 2 to available receivers RX-CH, RX24, RX18, and RX6 (e.g., present in the MRT 101). With multichannel systems, it may be possible to realize a technical implementation that does away with an arbitrary interconnectivity and replaces the arbitrary interconnectivity with a limited but adequate interconnectivity.

In addition to various implementation variants for existing MRT systems and local coil portfolios, the figures also depict a generalization of the approach enabling scaling to different MRT systems.

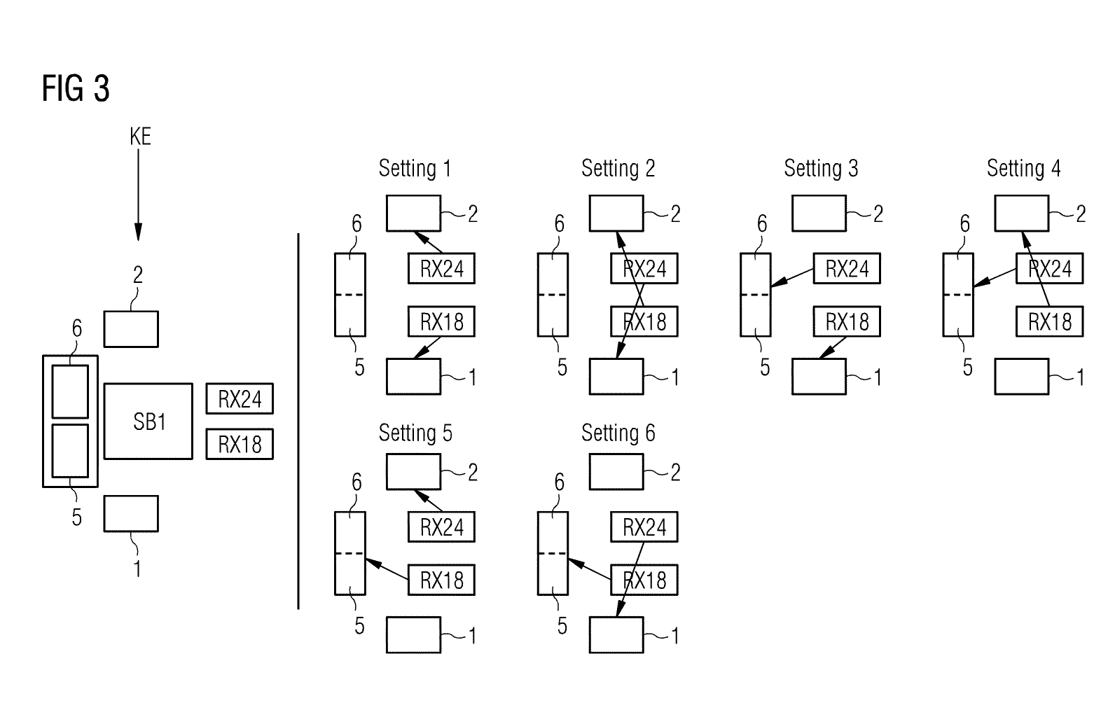

Some details of the settings depicted in FIGS. 3-6 are explained at greater length below (e.g., wherein the MRT connectors 5 and 6 may also only be connectable jointly to the same local coil).

For example, depicted on the left in FIG. 3 are patient couch MRT connectors 1, 2, 5, and 6 plugged in at the head end (KE) of a patient couch (e.g., 1 and/or 2 for one local coil each, and 5 and/or 6 in combination for a further, third local coil), two blocks RX24 (e.g., of 24 receivers RX-CH) and RX18 (e.g., of 18 receivers RX-CH) and a switching matrix SB1 for the connection (e.g., switchable by a controller of the MRT or of a local coil) of some or all contacts of MRT connectors 1, 2, 5, and 6 to some or all contacts of one or more of the receiver blocks RX18 and/or RX24.

Depicted on the right in FIG. 3 are six different “Settings” 1-6 (e.g., switch positions or use cases) of the switching matrix SB1 indicated only by arrows. The arrows in each represent one of the connections switched by switching matrix SB1 of some or all contacts of one of the MRT connectors 1, 2, 5, and 6 to some or all contacts of one or more of the two receiver blocks RX18 and RX24.

According to Setting 1, therefore, the receiver block RX24 may be switched to the MRT connector 2 by the switching matrix SB1, and the receiver block RX18 to the MRT connector 1, etc.

Depicted on the right in FIG. 4 are four different “Settings” 1-4 (e.g., switch positions or use cases) of the switching matrix SB1 indicated only by arrows. The arrows in each represent one of the connections switched by switching matrix SB1 of some or all contacts of one of the MRT connectors 1, 2, 5, and 6 to some or all contacts of one or more of the two receiver blocks RX18 and RX24 (e.g., of which one can only be connected to two of the three MRT connectors 1, 2, 5, and 6).

According to Setting 1, therefore, the receiver block RX24 may be switched to the MRT connector 2 by the switching matrix SB1, and the receiver block RX18 to the MRT connector 1, etc.

FIG. 5 depicts nine different “Settings” 1-9 (e.g., switch positions or use cases) of the switching matrix SB1 indicated only by arrows. The arrows in represent one of the connections switched by switching matrix SB1 of some or all contacts of one of the MRT connectors 1, 2, 5, and 6 to some or all contacts of one or more of the three receiver blocks RX18 (e.g., top), RX6, and RX18 (e.g., bottom).

According to Setting 1, the upper receiver block RX18 and the receiver block RX6 may be switched to the MRT connector 2 by the switching matrix SB1, while the lower receiver block RX18 is switched to the MRT connector 1.

According to Setting 2, the lower receiver block RX18 and the middle receiver block RX6 may be switched to the MRT connector 1 the switching matrix SB1, while the upper receiver block RX18 is switched to the MRT connector 2 (e.g., two of three receiver blocks are connected to the same MRT connector).

According to Setting 8, the upper receiver block RX18 may be switched to the MRT connector 2 by the switching matrix SB1, the lower receiver block RX6 may be switched to the MRT connector (e.g., connector group) 5 and 6, while the middle receiver block RX18 is switched to the MRT connector 1 (e.g., each of three receiver blocks is connected to a different MRT connector).

FIG. 6 depicts seven different “Settings” 1-7 (e.g., switch positions or use cases) of the switching matrix SB1 indicated only by arrows, with the arrows in each representing one of the connections switched by switching matrix SB1 of (e.g., some or all contacts of) one of the MRT connectors 1, 2, 5, and/or 6 to (e.g., some or all contacts of) one or more of the three receiver blocks RX18, RX6, and/or RX18.

According to Setting 1, the lower receiver block RX18 and the receiver block RX6 may be switched to the MRT connector 1 by the switching matrix SB1, while the upper receiver block RX18 is switched to the MRT connector 2.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description

1. A magnetic resonance tomography (MRT) system comprising:
a plurality of MRT connectors for connecting antenna elements to respective ones of a plurality of local coils,
wherein the antenna elements of a first one of the local coils connect to a first one of the MRT connectors are switched by a switching matrix to receivers of a first one of a plurality of receiver blocks, each the plurality of receiver blocks having a plurality of receivers.

2. The system of claim 1, wherein all of the antenna elements of the first one of the local coils connected to the first one of the MRT connectors are switched by the switching matrix to the receivers of a first one of a plurality of receiver blocks, and wherein all of the antenna elements of another of the local coils connected to another of the MRT connectors are switched by the switching matrix to receivers of another receiver block.

3. The system of claim 1, wherein fewer receivers are provided in the magnetic resonance tomography system than the total number of antenna elements provided by the local coils connectable to the MRT connectors.

4. The system of claim 1, wherein the switching matrix, the receivers, or the switching matrix and/or receivers are installed in the patient couch of the magnetic resonance tomography system.

5. The system of claim 1, wherein, out of fewer than all the MRT connectors connected to the switching matrix, only antenna elements that are connectable to said MRT connectors are switched to the receivers by the switching matrix.

6. The system of claim 1, wherein the switching matrix connects precisely one of the antenna elements to precisely one of the receivers by through-connecting in a DC-coupled, optical, inductive and/or decoupled manner.

7. The system of claim 1, wherein each of the receivers has one amplifier.

8. The system of claim 1, wherein some or all of the MRT connectors are patient couch connectors mounted on a patient couch, wherein the patient couch connectors are mounted at the head end, at the foot end of a patient couch, or a combination thereof.

9. The system of claim 1, wherein some or all of the MRT connectors are mounted on the housing of the MRT, at the bore of the MRT, or a combination thereof.

10. The system of claim 1, wherein the switching matrix, the receivers, or the switching matrix and the receivers are installed in a patient couch of the magnetic resonance tomography system at the head end, or at the end of the patient couch that is closer to the bore when the patient couch is withdrawn from the bore of the magnetic resonance tomography device.

11. The system of claim 1, wherein the switching matrix, the receivers, or the switching matrix and the receivers are installed in the patient couch of the magnetic resonance tomography system at the foot end or, at the end of the patient couch farther away from the bore when the patient couch is withdrawn from the bore of the magnetic resonance tomography device.

12. The system of claim 1, wherein the switching matrix, the receivers, or the switching matrix and the receivers are installed on the housing of the MRT, at the bore of the MRT, or a combination thereof.

13. The system of claim 1, wherein the receivers are arranged in one, two, three or more groups of receive blocks.

14. The system of claim 1, wherein the receivers are arranged in two, three, or more than three groups of receive blocks that all contain a different number of receivers.

15. The system of claim 1, wherein the receivers are arranged in two, three, or more than three groups of receive blocks that all contain an equal number of receivers.

16. The system of claim 1, wherein the receivers are arranged in three or more than three groups of receive blocks, wherein two of the groups contain a different number of receivers, and wherein two of the groups contain the same number of receivers.

17. The system of claim 1, wherein the receivers are arranged in groups of receive blocks, wherein at least one of the groups has fewer receivers than there are antenna elements that are connectable to at least one of the MRT connectors.

18. The system of claim 1, wherein the receivers are provided or arranged in groups of receive blocks, wherein at least one receive block has the same number of receivers as there are antenna elements that are connectable to at least one of the MRT connectors.

19. The system of claim 1, wherein the MRT connectors connected to the switching matrix have the same number of contacts, may be connected to the same number of antenna elements of local coils that are connectable to the MRT connectors, or a combination thereof.

20. The system of claim 1, wherein the MRT connectors connected to the switching matrix have a different number of contacts, are connectable to the same number of antenna elements of local coils, or a combination thereof.

21. The system of claim 1, wherein the MRT connectors connected to the switching matrix are connectable to a total of ninety-six antenna elements of local coils.

22. The system of claim 1, wherein out of the MRT connectors connected to the switching matrix, one of the MRT connectors are connectable to sixty-four, thirty-two, twenty-four, eighteen, sixteen, twelve, eight, four or two antenna elements of a local coil.

23. The system of claim 1, wherein out of the MRT connectors connected to the switching matrix, one is connectable to six receivers, connectable to eighteen receivers, connectable to twenty-four receivers, or a combination thereof.

24. The system of claim 1, wherein out of the MRT connectors connected to the switching matrix, each of the MRT connectors are connectable or connected to a different local coil.

25. The system of claim 1, wherein out of the MRT connectors connected to the switching matrix, two of the MRT connectors are connectable or connected to the same local coil.

26. The system of claim 1, wherein each of the antenna elements is connectable to any one of the receivers by the switching matrix, wherein each of the antenna elements is always connected to precisely one of the receivers at any point in time.

27. The system of claim 1, wherein the antenna elements are connected to one of the receivers by the switching matrix, wherein transmission therebetween is provided by frequency-division multiplexing, time-division multiplexing, or code-division multiplexing of signals of a plurality of antenna elements on a common transmission path.
